(19) United States
(12) Patent Application Publication
(10) Pub. No.: US 2011/0287168 A1
(43) Pub. Date: Nov. 24, 2011

(54) Wet Granulation System Comprising at Least One Ultrasonic Nozzle

(76) Inventor: Ferenc Buzsaky, Sodertalje (SE)

(21) Appl. No.: 13/061,166

(22) PCT Filed: Aug. 28, 2009
(86) PCT No.: PCT/SE2009/050974

§ 371 Date: May 20, 2011

Related U.S. Application Data
(60) Provisional application No. 61/092,862, filed on Aug. 29, 2008.

(51) Int. Cl. A61K 9/28; B05C 5/00

(52) U.S. Cl. 427/2.18; 118/24

(57) ABSTRACT

The present disclosure relates to a system for uniform distribution of a liquid binder onto a surface of particulate solids of at least one pharmaceutical product. The system comprises a substantially circular mixer, provided with rotating means in a lower part, configured to rotate the solids along a periphery of the mixer in a first rotational movement, and at least one ultrasonic nozzle connected to a feeding device providing the said liquid binder, and configured to distribute the liquid binder as droplets onto the surface of the solids during their rotational movement.

### TECHNICAL FIELD

The present invention relates to a system for uniform distribution of a liquid binder onto the surface of finely particulate solids of at least one pharmaceutical product.

The invention further relates to use of such a system, and to a method for uniform distribution of a liquid binder onto the surface of finely particulate solids.

### BACKGROUND OF THE INVENTION

Distribution of a liquid binder onto the surface of a pharmaceutical solids, finely particulate solids of a pharmaceutical product, so called wet granulation, is widely used within the pharmaceutical industry for the formulation of solid dosage forms. Wet granulation is a size enlargement process in which liquid binder is used to agglomerate solid particles. The particles in the pharmaceutical solids are bound together by the liquid is binder through capillary and viscous forces until drying where more permanent bonds are formed.

The granulation process enlarges particles of pharmaceutical solids and the thus enlarged particles are usually named granules. The granulation process changes physical and rehological properties of the pharmaceutical solids. The main reasons for granulation are to prevent segregation of constituents of pharmaceutical solids mixed of several pharmaceutical components, to enhance flow properties of the pharmaceutical solids, to improve the compaction characteristics of the pharmaceutical solids, to reduce dust and to densify the pharmaceutical solids. By changing the above mentioned characteristics of the pharmaceutical solids in an accurate way, handling and further processing of the pharmaceutical solids into such as pressing tablets in a tablet machine are improved. During the last decades there has been a considerable improvement in understanding the granulation process. Today, the granulation process is considered to be an example of pharmaceutical particle design were the desired attributes of the so produced granules are controlled in an accurate way by a combination of formulation variables (of the pharmaceutical solids and of the liquid binder) and of machine-dependent process parameters.

The main formulation variables affecting the quality of the produced granules are pharmaceutical solids particle size distribution, wetting of the solid by the liquid binder, solid solubility and characteristics and amount of the liquid binder. The knowledge of the granulation growth process is increasing rapidly and three main processes affecting granulation behaviour have been identified. The three main processes are identified as wetting and nucleation, consolidation and growth, and attrition and breakage. The researchers within the granulation area believe that an understanding of the identified processes will enable prediction of how formulation variables and process parameters together affect the produced granules.

In granulation processes, the main process variables affecting the quality of the granules is are the speed of the impeller in the mixer, the granulation time, the temperature and the method of addition of a liquid binder.

Improper granulation causes problems in down stream processes such as caking, segregation and poor tableting performance and therefore the granulation process is to be considered as a very important step in the production of solid dosage forms.

One key factor for the granulation process is the distribution and the droplet size of the binder liquid, having a major influence on wetting. The size of the droplets further affects the growth behaviour of the granules. If the liquid binder droplet size is much larger than the size of the primary particles, this results in growth by the immersion mechanisms instead of the distribution mechanism that results in production of over sized granules. Such over sized granules need to be chopped to smaller granules before use in further processes. To chop the granules, a chopper needs to be present in the mixer, and the chopping process is depending on the chopper speed and on shear forces to break down the over wetted lumps.

The method of liquid binder addition is thus very important for the quality of the produced granules. Conventionally, two main ways are used for addition of the liquid binder: pouring and spraying of the liquid binder to be distributed.

The pour-on method includes pouring of a liquid binder directly onto a moving bed of pharmaceutical solids without any liquid binder dispersion. Liquid binder distribution by the pour-on method is solely dependent on mechanical mixing which is why this method causes a very poor initial liquid binder distribution. This uneven liquid binder distribution causes local areas of high moisture content and superior growth while other areas remain ungranulated.

Although the pour-on method possesses some advantages including the ease of processing and the short process time, as a consequence of the above mentioned disadvantage with is uneven distribution of the liquid binder, this method is not applicable where accurate and even distribution of the liquid binder is critical to the quality of the thus produced granules.

The spray-on method provides a more accurate method for distribution of a liquid binder. The method involves liquid binder dispersion into droplets by passing a liquid binder through one or several nozzles at high pressure and high velocities. The droplets are sprayed under pressure onto a moving bed of pharmaceutical solids. To avoid over wetting, it is desirable to add the liquid binder slowly. With the spray on-method, the liquid binder is added slowly compared to the pour-on method. Further, to improve liquid binder distribution with the spray-on method small nozzles are used for spraying. Smaller nozzles, and hence smaller droplets, provide a better liquid binder distribution. Although the spray-on method has advantages compared to the pour-on method, it is possessed with limitations. Due to the need of the liquid binder to be added during pressure, the nozzles can be plugged up with the pharmaceutical solids to be granulated, and for this reason the orifice of the nozzle cannot be too small. The pressure cannot be too low, since it results in clogging of the nozzle and in the granulation time being too long.

Another disadvantage with the spray-on method is that the droplets show a wide size distribution, causing a wide granule size distribution. Further, the need of the liquid binder to be added under pressure results in loss of pharmaceutical solids in filters and on the walls of the equipment. As a result of the facts described above, it is difficult to control the droplets size. Increasing the pressure leads to smaller droplet size and higher flow rate but more over wetting due to dens impact of droplets. Decreasing the nozzle orifice decreases the liquid binder flow and increases the risk for clogging of the nozzle.

Further, a substantial pressure added to the liquid binder thus dispersing the pharmaceutical solids, results in the bottom and the wall of the container becoming wet. As a result, it is difficult to achieve uniform granules with narrow particle size distribution. If the pressure is reduced, the liquid binder droplets become larger with the drawback that humidification becomes uneven. Another problem is that the dispersed pharmaceutical solids stuck to the nozzle disturbing the spray pattern with uneven humidification as a is result. Further, the over pressure also create a dim of pharmaceutical solids clogging any filter unit connected to the granulation mixer, and it also decreases the yield for the granulation.

Yet another problem arises with some formulations, i.e. formulations containing different gelling polymers. A gelling polymer herein is defined as a polymer that is able to form a solid transient three-dimensional network that spans through a liquid medium. Examples of gelling polymers, which may be synthetic or natural, include polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol®); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; poly(ethylene oxide) (PEO); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques. Formulations comprising gelling polymers can be very sensitive to over wetting since the pharmaceutical solids swells during granulation and create lumps in different sizes which can be difficult to mill after drying, resulting in lower yield. Another critical factor is also the granulation time for these kinds of formulations. Long time generates larger lumps that can be difficult to dry and mill. These lumps tend to be very hard after the drying process, resulting in granules with low compressibility not being suitable for use in for example a tablet pressing process.

### THE OBJECT OF THE INVENTION

It is an object of the present invention to provide a system and a method for uniform distribution of a liquid binder onto the surface of finely particulate solids of at least one pharmaceutical product, solving the problems mentioned above.

### SUMMARY OF THE INVENTION

The object mentioned above is achieved by providing a system for uniform distribution of a liquid binder onto the surface of finely particulate solids of at least one pharmaceutical product. In preferred embodiments, finely particulate refers to a material having a mean particle, preferably measured by sieve analysis, of less than 250 μm, preferably less than 100 μm. The system comprises a substantially circular mixer, provided with rotating means in the lower part, arranged to enable the said solids to rotate along the periphery of the mixer in a first rotational movement, and at least one ultrasonic nozzle connected to a feeding device providing the liquid binder, and arranged to distribute the said liquid binder in the form of droplets onto the surface of the said solids during their rotational movement. By using an ultrasonic nozzle for distribution of the liquid binder in a very accurate way in the form of droplets not being under pressure, production of granules with good flow ability, small size distribution, porous structure and good compressibility are enabled. Since the thus produced granules are small and in a unitary size, there is no need for a chopper chopping the granules to smaller size, for producing small granules.

Ultrasonic atomization involves the formation of fine droplets by the vibration of a thin liquid binder film on a vibrating surface. The thus formed droplets are then ejected from the vibrating surface into the surrounding as a dense fog, falling down by gravity avoiding dens impact. If the pharmaceutical solids flow is sufficient high and a proper liquid binder flow is used, a short wetting time is obtained leading to improved control of the growth of the granules. Since the surface of the nozzle is vibrating no pharmaceutical solids is stuck to the nozzle and disturbing the spray pattern. Further, production of granules that preferably comprise gelling polymers with good flow ability, small size distribution, porous structure and good compressibility are enabled. Further, the use of ultrasonic nozzles in granulation processes also have an advantage regarding the size of the droplets, since the droplets are more even in size, and the size are controlled in a very accurate way by changing the amplitude—input energy, and the liquid binder flow.

According to at least one embodiment of the invention, the mixer is further provided with a conical surface in the upper part, arranged to enable the solids to rotate in at least a second rotational movement. The rotational axis of the first rotational movement being declined from the rotational axis of the second rotational movement.

By bringing the solids into a rotational movement, with rotational movements in several directions, the area of the solids are exposed to the liquid binder in an efficient way, resulting in even distribution of the liquid binder on the whole surface of each solid.

According to at least one embodiment of the invention, the size of the droplets is between 25 μm and 300 μm in diameter.

According to at least one embodiment of the invention, the flow rate of the liquid binder is between 10 g/min and 2000 g/min.

According to at least one embodiment of the invention, the temperature of the liquid binder is between 5° C. and 75° C.

According to at least one embodiment of the invention, two or more nozzles are comprised, said nozzles being arranged around the periphery of the mixer.

The invention further relates to use of such a system wherein at least one of the pharmaceutical products comprise a gelling polymer.

The invention further relates to a method for uniform distribution of a liquid binder onto is the surface of finely particulate solids of at least one pharmaceutical product in a mixer, comprising the steps:
bringing the solids into a first rotational movement along the periphery of the mixer,
distributing the said liquid binder in the form of droplets onto the surface of the finely particulate solids of the pharmaceutical product during their rotation, wherein said droplets have the same pressure as the surrounding air.

According to at least one embodiment of the invention, the solids are brought into a second rotational movement, the rotational axis of the first rotational movement being declined from the rotational axis of the second rotational movement.

Medicaments suitable for granulation in such a system include for example Seroquel™ (Quetiapine).

### BRIEF DESCRIPTION OF THE DRAWINGS

The present invention will now be described, for exemplary purposes, in more detail by way of embodiments and with reference to the enclosed drawings, in which:

FIG. 1 shows an ultrasonic atomizer used in a mixer with bottom drive impeller that is an embodiment of the present invention.

FIGS. 2, 3 and 4 show results from a comparative example with conventional granulation process compared to the invention.

FIGS. 2a-2e show tables over the result.

FIG. 3 shows particle size distribution after granulation.

FIG. 4 shows compressing profile and tablet hardness.

### DETAILED DESCRIPTION OF PREFERRED EMBODIMENTS

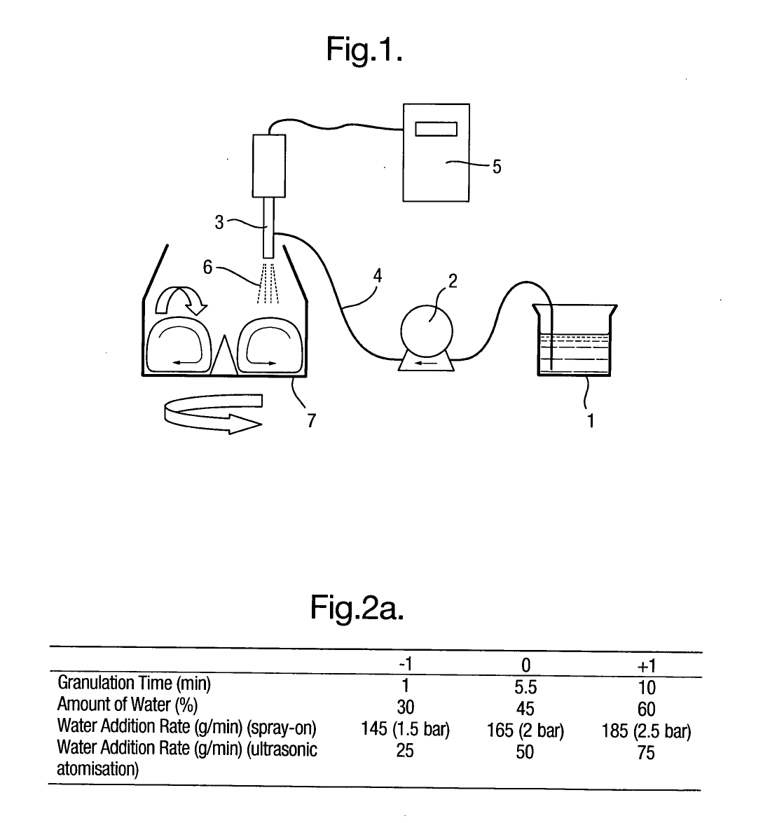

FIG. 1 shows an embodiment of the present invention. An ultrasonic atomizer nozzle 3 is used in a mixer 7 with rotating means, i.e. a bottom drive impeller. Fine particulate solids of at least one pharmaceutical product, i.e. in the form of pharmaceutical solids material, are added in a mixer 7, and an appropriate amount of liquid binder 1, i.e. an aqueous or an organic solution, is applied from above by an ultrasonic atomizer equipped with an atomizer nozzle 3.

During operation, the system works as follows. A fixed amount of granulation liquid binder 1 is supplied by means of a metering pump 2 (for example a gear pump) to a nozzle 3 via a tube 4. Appropriate ultrasonic vibrations are imparted to the nozzle 3 by a control unit 5 to discharge droplets 6 into a mixer 7. By using a gear pump together with an ultrasonic-unit, the flow of liquid binder 1 is controlled very accurately and thus, as a consequence, indirectly controlling the granule growth.

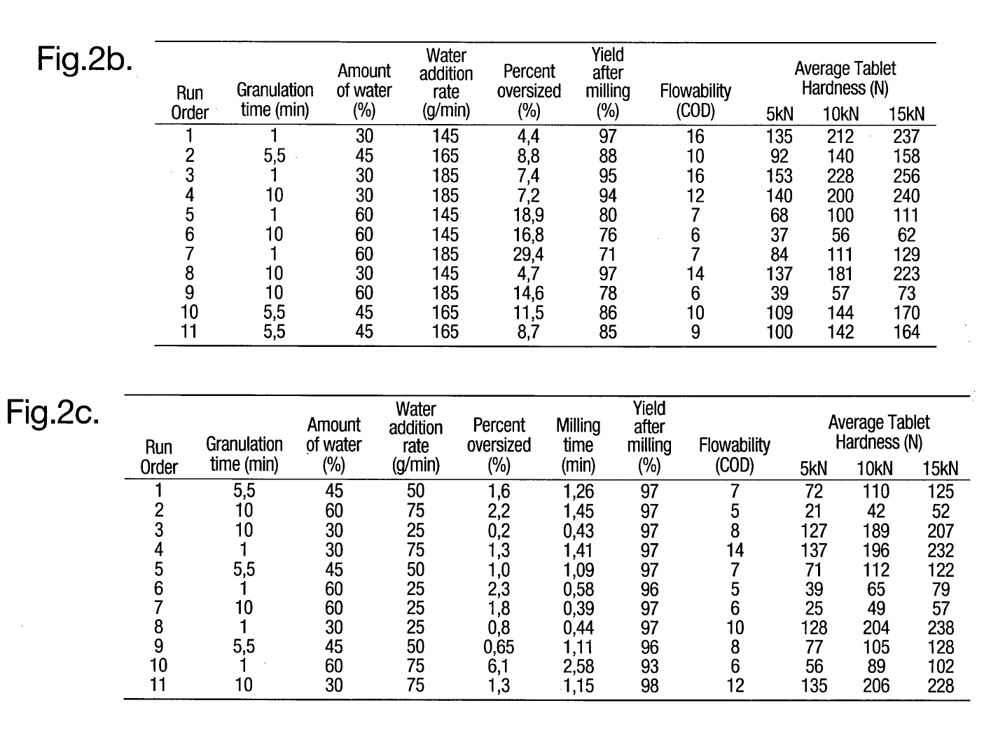

In FIGS. 2, 3 and 4 the results of an example evaluating the new granulation process compared to the spray-on method are shown. The example is performed with a formulation consisting of Hydroxyl-propyl-methyl cellulose (HPMC) 15000 cps and Poly-vinyl-pyrrolidone (PVP). As a granulation liquid binder water was used.

A factorial design was used to find the optimal conditions for the two methods concerning the process factors volume of liquid binder addition, granulation time and water addition rate with regard to the responses variables percent oversized (>1.6 mm), yield after milling, flow ability and tablet hardness. A factorial design involves the creation of a set of representative experiments where all factors are varied simultaneously and enables the extraction of a lot of information from a few experiments.

The parameter limits are shown in the table in FIG. 2a, and the total experimental design and the results achieved can be seen in the tables in FIG. 2b, showing data for the spray-on method, and in FIG. 2c, showing data for the ultrasonic method. FIG. 2d shows favourable granulation process parameters found for the different methods of addition of a liquid binder. FIG. 2e shows data over the granules produced in the experiment.

Experiments number 1 and 2 use spray-on methods and experiments 3 and 4 use ultrasonic atomisation.

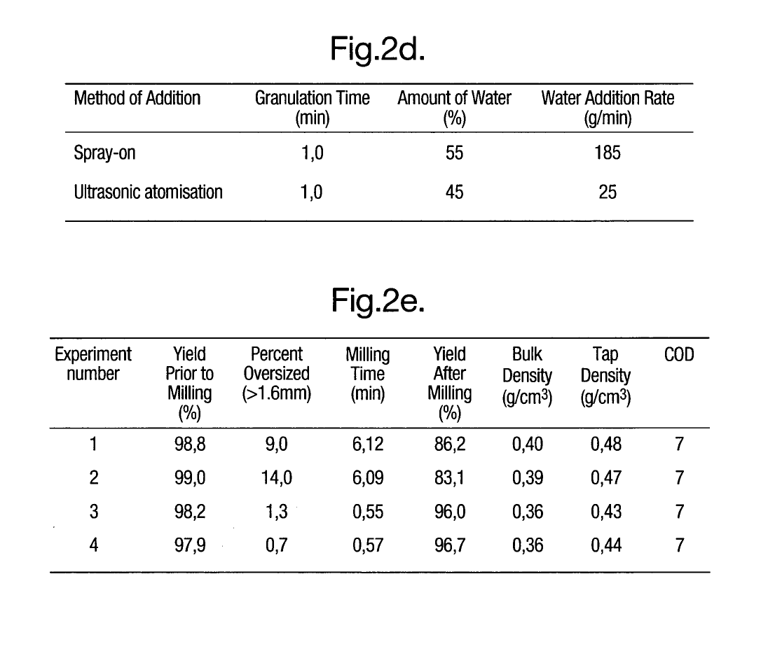

FIG. 3 shows the particle size distribution after granulation. The sieve analysis shows that when using ultrasonic atomisation the granules have more narrow distribution and are more homogenous in comparison to the granules produced with the spray-on method.

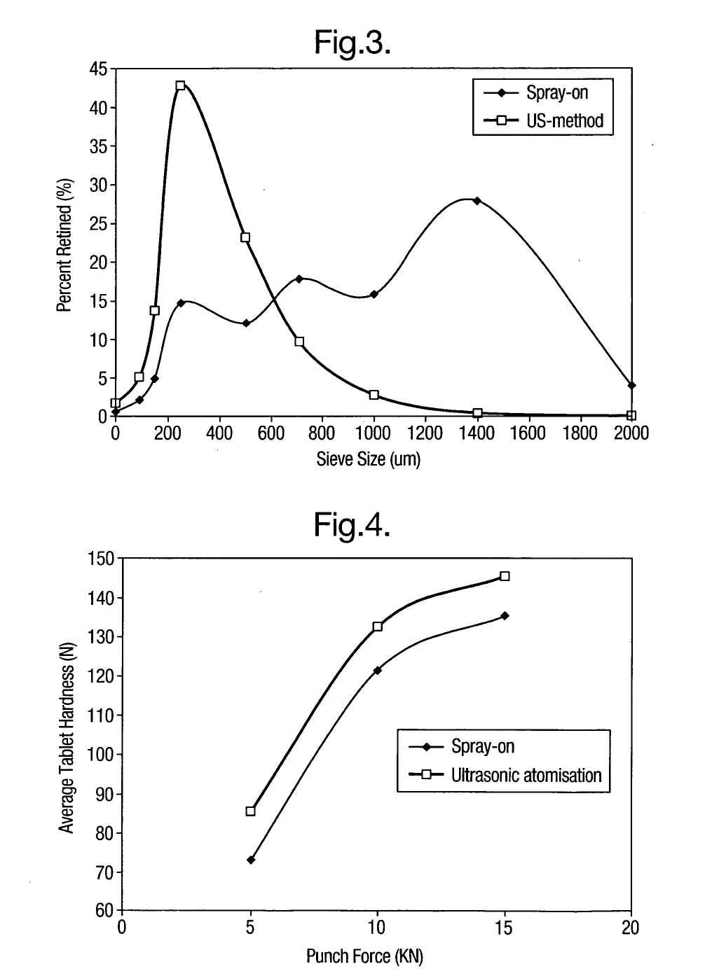

As can be seen in FIG. 4 the granules prepared by the new method accomplish tablets with the highest fracture resistance at all investigated punch forces. An explanation to this can be the higher bulk density of the granules produced by the spray-on method, see FIG. 2e. A high bulk density for powders of comparable true density is associated with a decreased porosity, which usually is connected to a decreased compactability.

The granulation was implemented in a high-shear mixer (Aeromatic-Fielder, GP-1). All granulations were preformed with a batch size of 1000 g, which corresponds to a fill level of approximately 40 percent. Prior to granulation the formulation components were blended in the mixer for 3 minutes at 250 rpm. For the granulation the impeller speed was set to 350 rpm, and the chopper speed was kept constant at 1000 rpm. When using the to ultrasonic atomisation method for granulation, the chopper was dismounted and not in use.

Further, it will be understood that the present invention is not limited to the described embodiments but can be modified in many different ways without departing from the scope of the appended claims.

1. A system for uniform distribution of a liquid binder onto a surface of particulate solids of at least one pharmaceutical product, comprising:
a substantially circular mixer, provided with rotating means in a lower part, configured to rotate the solids along a periphery of the mixer in a first rotational movement; and
at least one ultrasonic nozzle connected to a feeding device providing the liquid binder, and configured to distribute the liquid binder as droplets onto the surface of the solids during their rotational movement.

2. A system according to claim 1, wherein the mixer is further provided with a conical surface in the upper part, configured to rotate the solids in at least a second rotational movement, a rotational axis of the first rotational movement being declined from a rotational axis of the second rotational movement.

3. A system according to claim 1, wherein a size of the droplets is between 25 μm and 300 μm in diameter.

4. A system according to claim 1, wherein a flow rate of the liquid binder is between 10 g/min and 2000 g/min.

5. A system according to claim 1, wherein a temperature of the liquid binder is between 5° C. and 75° C.

6. A system according to claim 1, further comprising two or more nozzles, wherein the nozzles are arranged around the periphery of the mixer.

7. A system according to claim 1, wherein the at least one pharmaceutical product includes a gelling polymer.

8. A method for uniform distribution of a liquid binder onto a surface of particulate solids of at least one pharmaceutical product in a mixer, comprising the steps:
rotating the solids in a first rotational movement along a periphery of the mixer; and
distributing the liquid binder as droplets onto the surface of the particulate solids of the at least one pharmaceutical product during rotation, wherein a pressure of the droplets is the same as a pressure of the surrounding air.

9. A method according to claim 8, further comprising rotating the solids in a second rotational movement, wherein a rotational axis of the first rotational movement is declined from a rotational axis of the second rotational movement.
